(19) United States
(12) Patent Application Publication
(10) Pub. No.: US 2008/0241285 A1
(43) Pub. Date: Oct. 2, 2008

(54) Compositions and Methods to Treat Alopecia

(76) Inventor: Muhammed Majeed, Piscataway, NJ (US)

Correspondence Address: SABINSA CORPORATION, 70 ETHEL ROAD WEST, UNIT 6, PISCATAWAY, NJ, 08854, US

(21) Appl. No.: 12/060,558

(22) Filed: Apr. 1, 2008

Related U.S. Application Data
(60) Provisional application No. 60/909,483, filed on Apr. 1, 2007.

(51) Int. Cl. A61K 36/889; A61P 17/14

(52) U.S. Cl. 424/727

(57) ABSTRACT

Disclosed is a topical composition comprising therapeutically effective amounts of solubilized labdane diterpenoids in combination with freeze dried liquid endosperm of Cocos nucifera to (i) prevent the progression of alopecia; (ii) to treat alopecia and therapeutic methods thereof.

This application is a non-provisional filing of provisional U.S. patent application No. 60/909,483, filed on Apr. 1, 2007, the disclosure of which is herewith incorporated by reference.

### FIELD OF THE INVENTION

The invention in general relates to the treatment methods for alopecia. More specifically, the present invention relates to therapeutically effective amounts of labdane diterpenoids in combination with freeze dried liquid endosperm of Cocos nucifera to (i) prevent the progression of alopecia; (ii) to treat alopecia and therapeutic methods thereof.

### DESCRIPTION OF THE PRIOR ART

WO/1997/002041 titled, “Transdermal and oral treatment of androgenic alopecia” teaches the topical and oral treatment of hair loss, especially androgenic alopecia, by providing formulations that include anti-androgens, especially extracts of the saw palmetto plant, co-enzyme Q, and acetyl carnitine, and optionally stimulators of adenylate cyclase (forskolin) to stimulate hair growth, to increase the luster of hair, and to decrease hair graying.

JP09157136, titled “Hair restorer/Hair tonic” to Yokoyama Daizaburo on Jun. 17, 1997 discusses a combination of (A) tocotrienol and (B) forskolin or a derivative therefrom, or Coleus forskohlii root extract, and pref. furthermore, (C) at least one kind selected from among fatty acids, alcohols and derivatives therefrom, having odd-numbered carbon chains, respectively as hair restorers. The amounts of these ingredients to be formulated are (A) 0.01-10 wt. %, (B) 0.00001-10 wt. %, and (C) 0.01-20 wt. %. The weight ratio B/A is (1:100000) to (500:1). This hair restorer/hair tonic is used as an agent for external or internal use in the form of a homogeneous solution, lotion, gel, etc., by conventional means, and can also be served in the form of aerosol composition.

Based on the understanding that alopecia may be induced due to (i) Depression of physiological functions of scalp; (ii) Depression of metabolism function in hair follicles and hair bulb; (iii) Depression of the function of hair follicles due to male sex hormone action in sebaceous gland, hair follicles and hair root; (iv) Mental stress; (v) genetic factors and disease factors; (vi) aging of the hair matrix cells which suffer from the depression of cell division ability, cell differentiation ability and every metabolic activity including reduced blood flow in localized areas, leading to thinning of hair and epilation, the present inventors have invented a topical composition that comprises therapeutically effective amounts of a solubilized labdane diterpenoids in combination with freeze dried liquid endosperm of Cocos nucifera to (i) prevent the progression of alopecia; (ii) to treat alopecia and therapeutic methods thereof. The benefits of the liquid endosperm of coconuts have been well documented by the Coconut Development Board of India-Food and Agriculture Organization of the United Nations. Coconut water,

a) Contains organic compounds possessing growth promoting properties.
b) Help prevent inflammation (rashes and itches)
c) Help prevent bacterial, viral, and fungal infections.
d) Helps eliminate Candida yeast infections.
e) Helps eradicate eczema.
f) Helps keep skin soft and smooth.
g) Helps prevent dandruff.
h) Helps prevent premature aging and wrinkling of the skin.
i) Helps prevent skin cancer and other blemishes.
j) Helps prevent dry and chapped skin.

Accordingly, it is the principle object of the present invention to evaluate compositions comprising therapeutically effective amounts of solubilized labdane diterpenoids in combination with freeze dried liquid endosperm of Cocos nucifera to prevent the progression of alopecia/to treat alopecia.

It is also another object of the present invention to propose a method of preventing the progression of alopecia/treating alopecia through the use of a composition that provides the combined benefits of promoting metabolism including increased blood supply to hair matrix cells, promote hair growth and improving the ability of hair matrix cells to divide and differentiate, while preventing inflammation, microbial contamination, dandruff, blemishes, drying and chapping of the scalp leading to healthy hair.

It is yet another objective of the present invention to propose a method of preventing the progression of alopecia/treating alopecia through the topical application on the affected area of the scalp, a composition comprising therapeutically effective amounts of solubilized labdane diterpenoids in combination with freeze dried liquid endosperm of Cocos nucifera.

The present invention fulfills the aforesaid objectives and presents further related advantages.

### SUMMARY OF THE INVENTION

The present invention relates to the use of a solubilized form of labdane diterpenoids and freeze dried liquid endosperm of Cocos nucifera in the manufacture of a medicament for the treatment of alopecia, wherein the treatment method involves the steps of topically applying to the affected area of the scalp the said medicament comprising solubilized 2% w/w of forskolin and 5% freeze dried liquid endosperm of Cocos nucifera. Alternatively, the labdane diterpenoids may include derivatives and isomers of forskolin or combinations thereof.

### DETAILED DESCRIPTION OF THE PREFERRED EMBODIMENT

In the most preferred embodiment, the present invention relates to the use of a solubilized form of labdane diterpenoids and freeze dried liquid endosperm of Cocos nucifera in the manufacture of a medicament for the treatment of alopecia, wherein the treatment method involves the steps of topically applying to the affected area of the scalp the said medicament comprising solubilized 2% w/w of forskolin and 5% freeze dried liquid endosperm of Cocos nucifera. In alternate embodiments, the labdane diterpenoids may include derivatives and isomers of forskolin or combinations thereof.

The ability of the medicament comprising solubilized 2% w/w of forskolin and 5% freeze dried liquid endosperm of Cocos nucifera to promote hair growth and maintain hair health is discussed in suitable examples herein below.

C3H mouse dorsal hair is known to have a time synchronized hair cycle. From about 18-21 days of age and 47-95 days of age, the dorsal hairs are in the telogen phase. In this test, 8 weeks old male C3H/HeS1c mice whose hair cycle coincided with the telogen phase were used. The mice were grouped into 3 with each group having four mice. The hair on the back of each mouse was shaven with an electric shaver, care taken to ensure not to injure the skin. Approximately 4 cm² of the back of each mouse was shaven. Two hundred micro liters of test sample as elucidated herein below were applied on the shaven backs of the mice with application involving a careful massage of the area to ensure penetration and improved blood supply. The application was made thrice a day for three weeks test period. The test compositions included Minoxidil 1% (Control), 2% w/w of solubilized forskolin and 2% w/w of solubilized forskolin and 5% w/w of freeze dried liquid endosperm of Cocos nucifera. The composition comprising 2% w/w of solubilized forskolin and 5% w/w of freeze dried liquid endosperm of Cocos nucifera was efficacious in promoting the growth of fine soft hair in over 50% of the shaven area. The hair also exhibited overall tone, fine texture and color. This led the inventors to conclude that the topical composition comprising 2% w/w of solubilized forskolin and 5% w/w of freeze dried liquid endosperm of Cocos nucifera was efficacious in controlling the epilation caused due to various factors acting in alopecia.

The advantages of the present invention include
1. A therapeutic solution to the problem of alopecia with no side effects being wholly natural.
2. A comprehensive approach to the therapy of alopecia focusing on providing the combined benefits of promoting increased hair metabolism including blood supply to hair matrix cells, promoting hair growth and improving the ability of hair matrix cells to divide and differentiate, while preventing inflammation, microbial contamination, dandruff, blemishes, drying and chapping of the scalp leading to healthy hair.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. The use of a solubilized form of labdane diterpenoids and freeze dried liquid endosperm of Cocos nucifera in the manufacture of a medicament for the treatment of alopecia, wherein the treatment method involves the steps of topically applying to the affected area of the scalp thrice a day, the said medicament comprising solubilized 2% w/w of forskolin and 5% w/w freeze dried liquid endosperm of Cocos nucifera.

2. The use according to claim 1, wherein alopecia includes androgenic alopecia, alopecia greata, and alopecia totalis.

3. The use according to claim 1, wherein the solubilized labdane diterpenoid is deacetyl forskolin or its derivatives.

4. The use according to claim 1, wherein the solubilized labdane diterpenoid is isoforskolin or its derivatives.

5. The use according to claim 1, wherein the labdane diterpenoid is solubilized using randomly-methylated beta cyclodextrins (RAMBCD).
